(19) United States
(12) Patent Application Publication
(10) Pub. No.: US 2004/0086457 A1
(43) Pub. Date: May 6, 2004

(54) Product for testing for dysphagia

(76) Inventor: Wanda Kiiskila, Hershey, MI (US)

Correspondence Address: Flynn Thiel Boutell & Tanis, 2026 Rambling Road, Kalamazoo, MI, 49008-1631, US

(21) Appl. No.: 10/416,589

(22) PCT Filed: Nov. 7, 2001
(86) PCT No.: PCT/US01/51085

(30) Foreign Application Priority Data
Nov. 10, 2000 (US) 60247197

(51) Int. Cl. A61K 49/04

(52) U.S. Cl. 424/9.4; 424/9.411

(57) ABSTRACT

An orally ingestible baked radiopaque product comprising (a) fat, (b) sweetener, (c) moisturizing agent, (d) x-ray contrast agent, (e) flour and (f) optionally flavoring agent useful for evaluating swallowing food having a dry rood texture. The x-ray contrast agent is interspersed throughout the product by mixing it into the product prior to baking it. The product is used in the detection of dysphagia. A method and dough useful for preparing the product.

### FIELD OF THE INVENTION

This invention relates to an orally ingestible baked radiopaque product having a dry food texture that can be utilized for testing for dysphagia.

### BACKGROUND OF THE INVENTION

Dysphagia, or difficulty in swallowing, is a condition or symptom, which can be caused by many different conditions, injuries or diseases. Dysphagia can cause choking, aspiration of food or liquid into the lungs, aspiration pneumonia, incomplete administration of oral medication, and discomfort to the victims. These factors can at least discomfort a patient, but may also interfere with or complicate conventional treatment of patients when treating other coexisting disorders. Among the many contributions to or causes of dysphagia are head injuries, brain stem impairment, cerebral vascular accidents, Parkinsonism, Alzheimer's disease, muscular dystrophy, cerebral palsy, cancer, diabetes, medication side effects, advanced HIV infections, gastrointestinal disorders, gastro esophageal reflux disorder and brain tumors. It has been estimated that more than 10,000 patients a year choke to death as a result of dysphagia.

According to the American Speech Language Hearing Association (ASHA), an agency which accredits Speech-Language Pathologists and Audiologists, each year a large number of videofluoroscopic studies, commonly called Modified Barium Swallow (MBS) Studies or Cookie Studies, are administered in a radiological suite in order to assess patients swallowing abilities. A Speech-Language Pathologist (SLP) is a key person in this procedure often leading the team of experts in assessing a patient's swallowing abilities. The areas of the body assessed during these studies are the head, neck and upper chest regions with special emphasis on the oral cavity, pharyngeal cavity, laryngeal function, and esophageal motility. An MBS involves using a variety of foods and liquid textures and then observing the swallow while videotaping the motility x-ray. The purpose of an MBS is to find out why a person has difficulty swallowing and how to improve their swallowing abilities. Utilizing information obtained from the MBS, an SLP will assign postures, maneuvers and the least restrictive food or liquid textures that a patient can swallow in the safest manner. These MBS studies allow the assessment of airway protection during eating, management of chewing, tongue movement, laryngeal elevation, and timing of the entire system with each bite of food from the time it enters the mouth until it enters or exits the esophagus enroute to the stomach.

Liquid, semisolid and dry crumbly orally ingestible radiopaque materials containing barium sulfate are used in MBS procedures. Such materials are described in U.S. Pat. Nos. 5,024,240 and 5,976,084 and in Robertson et al, “A Strategy for Providing Food to the Patient with Neurologically Based Dysphagia”, Journal of the Canadian Dietetic Association, Vol. 54, No. 4 (Winter 1993), pp. 198-201, and Price et al, “A combined approach to the assessment of neurological dysphagia”, Clin. Otolaryngol. 12 (1987) pp. 197-201. However, none of these documents disclose any specific ingredients, let alone the ingredients utilized by Applicant to prepare an orally ingestible baked radiopaque product having a dry food texture.

Current MBS studies of dry textured food swallows is conducted utilizing cookies with barium sulfate paste applied to the top of the cookies in order to see the bolus formulation of dry texture substances. The cookies do not have a true dry food texture once the paste is applied to it. Applying paste changes the dry texture of the cookies into a mixed texture of dry and pudding combined. People with dysphagia have tremendous difficulty with the slightest change in food textures and mixing these textures can significantly alter the clinical or research data collected. Another disadvantage of using cookies pasted with barium sulfate for MBS studies is that only the mastication of the surface layer of barium sulfate on the product is visible during the studies and underlying crumbs may be lost in the system during mastication.

Furthermore, the prior art products used for MBS procedures have neither pleasant appearances nor tastes. Finally, they do not allow high accuracy or safety during assessment of chewing and swallowing.

### OBJECTS OF THE INVENTION

It is an object of the invention to provide an orally ingestible baked radiopaque product having a dry texture that can be used in the diagnosis of swallowing disorders, which affect the mastication and swallowing of foods having dry textures.

It is also an object of the invention to provide an orally ingestible baked radiopaque product that has a pleasant appearance and taste and that will help improve clinical data by allowing the SLP to obtain more accurate responses with dry textures of clients of any age.

It is also an object of the invention to provide a method of making an orally ingestible baked radiopaque product having a dry texture that can be used in the diagnosis of swallowing disorders, which affect the mastication and swallowing of foods having dry textures.

It is also an object of the invention to provide dough useful for making an orally ingestible baked radiopaque product having a dry texture that can be used in the diagnosis of swallowing disorders, which affect the mastication and swallowing of foods having dry textures.

It is another object of the invention to provide an orally ingestible baked radiopaque product that children are less likely to balk at taking.

It is yet another object of the invention to provide an orally ingestible baked radiopaque product having a dry food texture that can be used during MBS procedures wherein the mastication of the entire product is visible on x-ray and not just the surface layer, as is the case with prior art products.

It is still another object of the invention to improve over the limitation of the prior art to advance the diagnosis and treatment of swallowing disorders by providing an orally ingestible baked radiopaque product having a dry food texture wherein the x-ray contrast agent is interspersed throughout the product.

### SUMMARY OF THE INVENTION

This invention provides an ingestible baked radiopaque product having a dry food texture for testing for dysphagia, comprising:

a) fat,

b) sweetener,

c) moisturizing agent,

d) x-ray contrast agent,

e) flour and

f) optionally flavoring agent,

wherein the x-ray contrast agent is interspersed throughout the product.

A preferred ingestible baked radiopaque product is a cookie comprising:

a) fat

b) sweetener

c) moisturizer

d) x-ray contrast agent

e) flour and

f) optionally flavoring agent,

wherein the x-ray contrast agent is interspersed throughout the cookie.

Also provided is a method for testing a patient for dysphagia which comprises subjecting said patient to MBS studies utilizing an ingestible baked radiopaque product having a dry food texture comprising:

a) fat,

b) sweetener,

c) moisturizing agent,

d) x-ray contrast agent,

e) flour and

f) optionally flavoring agent,

wherein the x-ray contrast agent is interspersed throughout the product.

Also provided is a preferred method for testing a patient for dysphagia comprises subjecting said patient to MBS studies utilizing a radiopaque cookie having a dry food texture comprising:

a) fat,

b) sweetener,

c) moisturizing agent,

d) x-ray contrast agent,

e) flour and

f) optionally flavoring agent,

wherein the x-ray contrast agent is interspersed throughout the cookie.

Further provided is a method for making an orally ingestible baked radiopaque product having a dry food texture which comprises (1) mixing a) fat, b) sweetener, c) a moisturizing agent, d) a x-ray contrast agent, e) flour and (f) optionally flavoring agent to prepare dough (2) forming the dough into a shape adapted for oral ingestion and (3) baking the shaped dough.

Still further provided is a method of making a cookie having a dry food texture which comprises (1) mixing a) butter-flavored shortening, b) sugar, c) an egg yolk, d) pure vanilla extract, e) flour and f) barium sulfate to prepare dough (2) forming the dough into a cookie shape and (3) baking the shaped dough.

Dough comprising:

a) fat,

b) sweetener,

c) moisturizing agent,

d) x-ray contrast agent,

e) flour and

f) optionally flavoring agent.

Cookie Dough comprising:

a) fat,

b) sweetener,

c) moisturizer,

d) x-ray contrast agent,

e) flour and

f) optionally flavoring agent.

### DETALED DESCRIPTION OF THE INVENTION

The term “contrast agent” means any edible insoluble radiopaque material, which provides a positive contrast for x-ray procedures. The preferred contrast agent is powdered barium sulfate, a commercially available product.

The term “cup” means a volume of about 250 milliliters.

The term “dry food texture” means food having no or little moisture content. For example, cookies and foods of similar texture.

The term “fat” means butter, margarine, shortening” and mixtures thereof. Non liquid fat is preferred. Shortening is most preferred.

The term “egg” includes the eggs as well as egg yolks. It may be a small, medium, large or extra large size egg. Yolks from extra large size eggs are preferred.

The term “flavoring agent” means any flavoring agent that is known in the baking art for flavoring pastries. For example flavoring extracts such as pure vanilla extract, lemon extract, lemon flavored drink mix, almond extract; cocoa powder, etc. While the flavoring agent makes the product more favorable, it is not required.

The term “flour” means any flour that can be utilized to make baked pastries, crackers etc. Examples include white flour, wheat flour, graham flour, potato flour, rice flour and mixtures thereof.

The term “moisturizing agent” means eggs, milk, water and mixtures thereof.

The term “moisture powdered dough” means dough with a minimal amount of moisture and that has a non-sticky texture that allows it to be pressed into a mold.

The term “shortening” means a non-liquid edible fat that may be either flavored or unflavored. Butter flavored shortening is a preferred shortening.

The term “sweetener” means a sugar or a sugar substitute that does not deteriorate when subjected to baking temperatures. It may be in liquid, powdered or granulated forms. Powdered or granulated forms are preferred.

Temperatures as used herein are in degrees Fahrenheit.

The specific ratios of ingredients used to prepare the orally ingested baked radiopaque product of this invention can readily be determined and adjusted by those skilled in the art to vary the texture and taste of the material. The amount of x-ray contrast agent used in the dough should be sufficient to make the product radiopaque when the person who has ingested it is subjected to x-ray studies. For example, about ⅓ cup, preferably about ½ cup can be used in a batch of dough containing about 2½ cups of total ingredients. Also, the amount of product to be administered per study can readily be determined by those skilled in the art of administering MBS studies utilizing methods well known in the art.

This invention can be embodied in any baked food product that is in a shape adapted for easy oral ingestion such as cookies, crackers and other pastries that have a dry food texture as is demonstrated by the following examples. Ingredients, other than a-c, e and f defined above, that are utilized in baked product recipes, can be utilized in the products of this invention as long as the product retains its taste and dry food texture.

### EXAMPLE 1

### Butter Cookies

a) ½ cup butter flavored shortening

b) ½ cup sugar

c) 1 extra large egg yolk

d) ¼ tsp. pure vanilla extract

e) 1 cup of all purpose white flour

f) ½ cup barium sulfate

The butter flavored shortening is beaten with an electric mixer until it is fluffy. Sugar is added to the shortening and the mixture is beaten with an electric mixer until fluffy. Then the egg yolk and vanilla extract are beaten into the fluffy mixture with an electric mixer to provide a moist mixture. The flour and barium sulfate are mixed in a separate bowl and then mixed with the moist mixture with an electric mixer until blended. The blended dough is then scooped by 1-tablespoon amounts, poured pressed into cookie molds on an ungreased cookie sheet. During the pressing process, the dough is spread, flattened, pressed and shaped into the mold in to a ¼ inch thick cookies. The mold is removed and the cookies are baked at 375° F. for about five minutes to provide approximately 30 cookies.

### EXAMPLE 2

### Rice Flour Cookies

a) 1 cup butter, room temperature

b) ¼ cup powdered sugar

c) ¼ cup granulated sugar

d) 1 tsp. vanilla

e) ⅓ cup rice flour

f) ½ cup barium sulfate

g) ½-⅔ cup flour

Preheat oven to 325° F. In a large bowl, beat together butter, powdered sugar, granulated sugar and vanilla until light and fluffy. Add rice flour and barium and flour. The dough is worked by hand until smooth and no longer crumbly and then patted evenly into a buttered pan. The dough is then baked 35-45 minutes or until light brown and still somewhat springy to the touch. Cool 15 minutes. When slightly warm, remove from pan to obtain the title cookies.

### EXAMPLE 3

### Brown Sugar Cookies

a) 1 cup of all purpose white flour

b) 1 cup of butter

c) ½ tsp. vanilla

d) 1 tsp. baking powder

e) ½ cup brown sugar

f) ½ cup barium sulfate

Cream butter, gradually add brown sugar and vanilla. Add flour, baking powder and barium sulfate. Turn dough onto lightly floured surface and kneed lightly. Roll dough in a rectangular shape of about ¼ inch thickness. Cut into diagonal slices, like diamonds. Bake on cookie sheets in 350° F. oven for 20-25 minutes and then cool on wire racks to yield the title cookies.

### EXAMPLE 4

### Chocolate Shortbread

a) ½ cup butter, room temperature

b) ¼ cup packed brown sugar

c) ¼ tsp. vanilla

d) 1 cup (6 oz.) semi-sweet chocolate pieces (melted)

e) ½ cup flour

f) ½ cup barium sulfate

Preheat oven to 300° F. In a bowl, beat butter, brown sugar and vanilla until light and fluffy. Beat in melted chocolate to provide a moist mixture. Blend flour and barium sulfate. Add flour mixture to moist mixture and blend well. Press dough into ungreased molds. Impress edges of dough with a fork. Bake 45-50 minutes or until set and almost firm to touch. Cool in pan to yield the title cookies.

### EXAMPLE 5

### Crisp Sugar Cookies

a) ½ cup butter

b) 1 cup sugar

c) ½ cup sugar

d) 1 teaspoon vanilla

e) 1 egg

f) ½ cup flour

f) ½ cup barium sulfate

g) 2 teaspoons baking powder

h) ½ teaspoon salt

i) 2 teaspoons sugar+1 teaspoon of cinnamon mixture

j) egg white

Mix in order as written a-h. Make tablespoon sized balls and place on an ungreased cookie sheet and press into flattened shapes. Wash with egg white and sprinkle with mixture of 2 teaspoons of sugar+1 teaspoon of cinnamon. Bake at 350° F. for 8-10 minutes to yield title cookies.

It will be understood that the embodiments of the invention, which have been described, are merely illustrations of a few of the applications of the principles of the present invention. Numerous modifications may be made by those skilled in the art without departing from the true spirit and scope of the invention.

I claim:

1. An orally ingestible baked radiopaque product having a dry food texture for testing for dysphagia, comprising:
a) fat,
b) sweetener,
c) moisturizing agent,
d) x-ray contrast agent,
e) flour and
f) optionally flavoring agent,
wherein the x-ray contrast agent is interspersed throughout the product.

2. A product according to claim 1, wherein the x-ray contrast agent is barium sulfate.

3. A cookie comprising:
a) fat,
b) sweetener
c) moisturizer,
d) x-ray contrast agent,
e) flour and
f) optionally flavoring agent,
wherein the x-ray contrast agent is interspersed throughout the product.

4. A cookie according to claim 3, wherein the contrast agent is barium sulfate and the cookie contains a flavoring agent.

5. A cookie according to claim 4, wherein the fat is butter flavored shortening, the sweetener is sugar, the moisturizer is an extra large egg yolk and flour is all purpose white flour.

6. A method for testing a patient for dysphagia, which comprises, subjecting said patient to MBS studies utilizing an ingestible baked radiopaque product having a dry food texture comprising:
a) fat,
b) sweetener,
c) moisturizing agent,
d) x-ray contrast agent,
e) flour and
f) optionally flavoring agent,
wherein the x-ray contrast agent is interspersed throughout the product.

7. A method according to claim 6, wherein the x-ray contrast agent is barium sulfate and the product contains a flavoring agent.

8. A method according to claim 6, wherein the orally ingestible baked radiopaque product is a cookie comprising:
a) fat,
b) sweetener,
c) moisturizer,
d) x-ray contrast agent,
e) flour and
f) optionally flavoring agent.

9. A method according to claim 8, wherein the x-ray contrast agent is barium sulfate and the product contains a flavoring agent.

10. A method according to claim 9, wherein the fat is butter flavored shortening, the sweetener is sugar, the moisturizer is a egg yolk, the flavoring agent is vanilla extract and the flour is all purpose white flour.

11. A method for making an orally ingestible baked radiopaque product having a dry food texture which comprises (1) mixing a) fat, b) sweetener, c) moisturizing agent, d) x-ray contrast agent, e) flour and (f) optionally flavoring agent to prepare dough (2) forming the dough into a shape adapted for oral ingestion and (3) baking the shaped dough.

12. A method according to claim 11, wherein the x-ray contrast agent is barium sulfate and the dough contains a flavoring agent.

13. A method according to claim 11, wherein the orally ingestible baked radiopaque product is a cookie, which comprises (1) mixing a) fat, b) sweetener, c) moisturizer, d) x-ray contrast agent, e) flour and f) optionally flavoring agent to prepare a dough, (2) forming the dough into a cookie shape and (3) baking the shaped dough.

14. A method according to claim 13, wherein the x-ray contrast agent is barium sulfate and the cookie contains a flavoring agent.

15. A method according to claim 14, wherein the fat is butter flavored shortening, the sweetener is sugar, the moisturizer is an extra large egg yolk, the flour is all purpose white flour and the flavoring agent is vanilla extract.

16. Dough comprising:
a) fat,
b) sweetener,
c) moisturizing agent,
d) x-ray contrast agent,
e) flour and
f) optionally flavoring agent.

17. Dough according to claim 16, wherein the x-ray contrast agent is barium sulfate and the dough contains a flavoring agent.

18. Cookie dough according to claim 17, wherein the fat is butter flavored shortening, the sweetener is sugar, the moisturizing agent is an extra large egg yolk, the flavoring agent is vanilla extract and the flour is white all purpose flour.
